(19) United States
(12) Patent Application Publication
(10) Pub. No.: US 2026/0279573 A1
(43) Pub. Date: Sep. 17, 2026

(54) SYSTEMS AND METHODS FOR REFINING DISEASE DEFINITIONS USED TO CLASSIFY PROFILES

(71) Applicant: AML JV, LLC, Aurora, CO (US)

(72) Inventors: Justin DALE, Aurora, CO (US); Frank MARKSON, Aurora, CO (US); Brett STEVENS, Aurora, CO (US); Austin GILLEN, Aurora, CO (US); Md Nazmul ISLAM, Aurora, CO (US)

(73) Assignee: AML JV, LLC, Aurora, CO (US)

(21) Appl. No.: 19/562,306

(22) Filed: Mar. 10, 2026

Related U.S. Application Data
(60) Provisional application No. 63/770,226, filed on Mar. 11, 2025.

(51) Int. Cl. G16H 50/20; G16H 10/60; G16H 70/60

(52) U.S. Cl. CPC G16H 50/20; G16H 10/60; G16H 70/60

(57) ABSTRACT

Systems and methods for refining criteria associated with a disease to improve disease diagnosis at earlier stages of disease progression can include one or more processors configured to receive disease definition data identifying a plurality of attributes associated with a disease, each attribute mapped to at least one biomarker; generate, for each treatment profile, a normalized biomarker representation comprising quantified expression levels of the biomarkers; determine an initial diagnostic configuration comprising marker values defining classification thresholds for the attributes; execute a refinement protocol that iteratively evaluates misclassification of the treatment profiles by comparing the normalized biomarker representation to the classification thresholds; in response to the refinement protocol, generate an updated diagnostic configuration comprising updated marker values that reduce misclassification; and store the updated diagnostic configuration in non-transitory memory accessible by a diagnostic system configured to classify subsequently received treatment profiles.

### CROSS-REFERENCE TO RELATED APPLICATIONS

This application claims priority to U.S. Provisional Patent Application Ser. No. 63/770,226, filed Mar. 11, 2025, which is incorporated by reference in its entirety.

### TECHNICAL FIELD

This application relates generally to systems and methods for refining disease diagnosis definitions used to identify and analyze various medical conditions and, in some implementations, to techniques for utilizing patient data in models to increase the accuracy of disease diagnosis definition criteria.

### BACKGROUND

Clinicians traditionally rely on disease definitions established by organizations to guide them when diagnosing individuals as having a given disease. But these traditional disease definitions can rely on standards established by researchers that vary for a variety of reasons. For example, a given researcher or research organization developing a definition can over-index on a particular aspect of an individual's physiology believed to establish a biological target such as a protein or an enzyme that plays a role in the disease's progression. This over-indexing can subsequently bias aspects of the disease definition, resulting in a set of criteria that, when implemented, are too broad (result in overdiagnosis of the disease) or too narrow (result in underdiagnosis of the disease). As a result, definitions originating from different organizations can set different thresholds that result in inconsistent disease diagnosis that, in some cases, lead to oversights during diagnosis.

### SUMMARY

For the aforementioned reasons, there is a need for systems and methods that can refine disease definitions (e.g., through the use of models and/or the like) to determine updated threshold values indicative of the disease as increasing amounts of patient data become available for analysis. The systems and methods described herein address this need by implementing techniques to refine disease definitions as increasing amounts of patient data become available. For example, a system (referred to as an analytics server) can receive an input indicative of a set of attributes associated with a given disease definition. Each attribute can indicate a set of one or more marker values that are used to diagnose the disease with a first degree of precision. The system can then identify a dataset including treatment profiles associated with individuals who have been diagnosed with the disease and refine the definitions to determine an updated set of marker values that indicate a second degree of precision based on the dataset. Once updated, the marker values can be provided to configure diagnostic systems to classify subsequently-received treatment profiles, thereby reducing or eliminating overdiagnosis or underdiagnosis associated with the original disease definition. This, in turn, can allow for quicker identification of individuals having the disease (increasing the chances of better health short- and long-term health outcomes for the individuals than would be possible with later diagnosis) while reducing the chance that individuals that do not have the disease will be subjected to unnecessary and possibly invasive testing for the disease.

In an embodiment, a system for refining criteria associated with a disease to improve disease diagnosis at earlier stages of disease progression can include one or more processors, which can be configured to receive disease definition data identifying a plurality of attributes associated with a disease, each attribute mapped to at least one biomarker. The system can generate, for each treatment profile of a plurality of treatment profiles, a normalized biomarker representation comprising quantified expression levels of the biomarkers. The system can determine an initial diagnostic configuration comprising a set of marker values defining classification thresholds for the plurality of attributes. The system can execute a refinement protocol that iteratively evaluates misclassification of the treatment profiles by the initial diagnostic configuration by comparing the normalized biomarker representation to the classification thresholds. In response to the refinement protocol, the system can generate an updated diagnostic configuration comprising an updated set of marker values that reduce misclassification relative to the initial diagnostic configuration. The system can store the updated diagnostic configuration in a non-transitory memory accessible by a diagnostic system, wherein the diagnostic system is configured to classify subsequently received treatment profiles using the updated diagnostic configuration.

In some implementations, the system can receive, via a network interface, an indication identifying one or more misclassified treatment profiles and a corresponding ground-truth label for each misclassified treatment profile. In some implementations, the system can write the indication and the ground-truth label for each misclassified treatment profile to an audit log data structure stored in the non-transitory memory. In some implementations, the system can execute the refinement protocol using the audit log data structure to generate the updated diagnostic configuration.

In some implementations, the initial diagnostic configuration comprises a first ruleset comprising a first set of marker values defining a first transition point for classifying a treatment profile into a first disease state and a second ruleset comprising a second set of marker values defining a second transition point for classifying a treatment profile into a second disease state. In some implementations, the first ruleset and the second ruleset are stored as versioned configuration records that are selectable by the diagnostic system at runtime.

In some implementations, the refinement protocol comprises, for each attribute, computing a marker deviation between the first set of marker values and the second set of marker values and storing the marker deviation in a deviation table comprising (i) an attribute identifier, (ii) a biomarker identifier, and (iii) a deviation value. In some implementations, the refinement
protocol comprises generating the updated set of marker values by applying the marker deviations from the deviation table to produce an updated ruleset that, when executed by the diagnostic system, reduces a misclassification rate for the plurality of treatment profiles relative to the initial diagnostic configuration.

In some implementations, the disease definition data comprises a first set of attributes associated with a first organization and a second set of attributes associated with a second organization. In some implementations, the system can generate the initial diagnostic configuration by producing a merged configuration record that (i) maps each attribute to a selected marker value chosen from a first set of marker values or a second set of marker values and (ii) stores, for each selected marker value, provenance metadata identifying the organization from which the selected marker value originated.

In some implementations, the refinement protocol comprises generating, for each treatment profile, a normalized biomarker feature vector comprising normalized expression levels corresponding to the attributes in the merged configuration record. In some implementations, the refinement protocol comprises comparing each marker deviation to the normalized biomarker feature vectors across the plurality of treatment profiles to compute a precision metric for the merged configuration record. In some implementations, the refinement protocol comprises generating the updated set of marker values by modifying the merged configuration record until the precision metric satisfies a target precision threshold, and storing the updated diagnostic configuration as a new version of the merged configuration record.

In some implementations, the refinement protocol comprises, for each attribute, generating a time-series record of biomarker expression levels for at least a subset of the treatment profiles. In some implementations, the refinement protocol comprises determining a change pattern by computing, from the time-series record, at least one rate-of-change value for the biomarker expression level over a defined time window. In some implementations, the refinement protocol comprises generating the updated set of marker values based on the rate-of-change value such that the updated diagnostic configuration classifies subsequent treatment profiles using both (i) a transition-point marker value and (ii) a change-pattern threshold derived from the rate-of-change value.

In some implementations, the system can determine correlations between a subset of the attributes across the plurality of treatment profiles by generating a correlation matrix data structure storing correlation coefficients indexed by attribute identifiers. In some implementations, the system can identify at least one different attribute based on correlation coefficients in the correlation matrix satisfying a selection criterion stored in memory. In some implementations, the system can update the plurality of attributes by updating an attribute registry that defines which biomarkers are ingested into the normalized biomarker representation. In some implementations, in response to updating the attribute registry, the system can regenerate the initial diagnostic configuration by generating a new versioned configuration record that includes marker values for the at least one different attribute.

In another embodiment, a method for refining criteria associated with a disease to improve disease diagnosis at earlier stages of disease progression can be performed, for example, by one or more processors coupled to non-transitory memory. The method can include receiving disease definition data identifying a plurality of attributes associated with a disease, each attribute mapped to at least one biomarker. The method can include generating, for each treatment profile of a plurality of treatment profiles, a normalized biomarker representation comprising quantified expression levels of the biomarkers. The method can include determining an initial diagnostic configuration comprising a set of marker values defining classification thresholds for the plurality of attributes. The method can include executing a refinement protocol that iteratively evaluates misclassification of the treatment profiles by the initial diagnostic configuration by comparing the normalized biomarker representation to the classification thresholds. The method can include, in response to the refinement protocol, generating an updated diagnostic configuration comprising an updated set of marker values that reduce misclassification relative to the initial diagnostic configuration. The method can include storing the updated diagnostic configuration in a non-transitory memory accessible by a diagnostic system, wherein the diagnostic system is configured to classify subsequently received treatment profiles using the updated diagnostic configuration.

In some implementations, the method can include receiving, via a network interface, an indication identifying one or more misclassified treatment profiles and a corresponding ground-truth label for each misclassified treatment profile. In some implementations, the method can include writing the indication and the ground-truth label for each misclassified treatment profile to an audit log data structure stored in the non-transitory memory. In some implementations, the method can include executing the refinement protocol using the audit log data structure to generate the updated diagnostic configuration.

In some implementations, the initial diagnostic configuration comprises a first ruleset comprising a first set of marker values defining a first transition point for classifying a treatment profile into a first disease state and a second ruleset comprising a second set of marker values defining a second transition point for classifying a treatment profile into a second disease state. In some implementations, the first ruleset and the second ruleset are stored as versioned configuration records that are selectable by the diagnostic system at runtime.

In some implementations, the refinement protocol comprises, for each attribute, computing a marker deviation between the first set of marker values and the second set of marker values and storing the marker deviation in a deviation table comprising (i) an attribute identifier, (ii) a biomarker identifier, and (iii) a deviation value. In some implementations, the refinement protocol comprises generating the updated set of marker values by applying the marker deviations from the deviation table to produce an updated ruleset that, when executed by the diagnostic system, reduces a misclassification rate for the plurality of treatment profiles relative to the initial diagnostic configuration.

In some implementations, the disease definition data comprises a first set of attributes associated with a first organization and a second set of attributes associated with a second organization. In some implementations, determining the initial diagnostic configuration further comprises generating the initial diagnostic configuration by producing a merged configuration record that (i) maps each attribute to a selected marker value chosen from a first set of marker values or a second set of marker values and (ii) stores, for each selected marker value, provenance metadata identifying the organization from which the selected marker value originated.

In some implementations, the refinement protocol comprises generating, for each treatment profile, a normalized biomarker feature vector comprising normalized expression levels corresponding to the attributes in the merged configuration record. In some implementations, the refinement protocol comprises comparing each marker deviation to the normalized biomarker feature vectors across the plurality of treatment profiles to compute a precision metric for the merged configuration record. In some implementations, the refinement protocol comprises generating the updated set of marker values by modifying the merged configuration record until the precision metric satisfies a target precision threshold, and storing the updated diagnostic configuration as a new version of the merged configuration record.

In some implementations, the refinement protocol comprises, for each attribute, generating a time-series record of biomarker expression levels for at least a subset of the treatment profiles. In some implementations, the refinement protocol comprises determining a change pattern by computing, from the time-series record, at least one rate-of-change value for the biomarker expression level over a defined time window. In some implementations, the refinement protocol comprises generating the updated set of marker values based on the rate-of-change value such that the updated diagnostic configuration classifies subsequent treatment profiles using both (i) a transition-point marker value and (ii) a change-pattern threshold derived from the rate-of-change value.

In some implementations, the method can include determining correlations between a subset of the attributes across the plurality of treatment profiles by generating a correlation matrix data structure storing correlation coefficients indexed by attribute identifiers. In some implementations, the method can include identifying at least one different attribute based on correlation coefficients in the correlation matrix satisfying a selection criterion stored in memory. In some implementations, the method can include updating the plurality of attributes by updating an attribute registry that defines which biomarkers are ingested into the normalized biomarker representation. In some implementations, the method can include, in response to updating the attribute registry, regenerating the initial diagnostic configuration by generating a new versioned configuration record that includes marker values for the at least one different attribute.

In yet another embodiment, a non-transitory computer-readable medium can store instructions thereon that, when executed by at least one processor, cause the at least one processor to receive disease definition data identifying a plurality of attributes associated with a disease, each attribute mapped to at least one biomarker. The non-transitory computer-readable medium can store instructions that cause the at least one processor to generate, for each treatment profile of a plurality of treatment profiles, a normalized biomarker representation comprising quantified expression levels of the biomarkers. The non-transitory computer-readable medium can store instructions that cause the at least one processor to determine an initial diagnostic configuration comprising a set of marker values defining classification thresholds for the plurality of attributes. The non-transitory computer-readable medium can store instructions that cause the at least one processor to execute a refinement protocol that iteratively evaluates misclassification of the treatment profiles by the initial diagnostic configuration by comparing the normalized biomarker representation to the classification thresholds. The non-transitory computer-readable medium can store instructions that cause the at least one processor to, in response to the refinement protocol, generate an updated diagnostic configuration comprising an updated set of marker values that reduce misclassification relative to the initial diagnostic configuration. The non-transitory computer-readable medium can store instructions that cause the at least one processor to store the updated diagnostic configuration in a non-transitory memory accessible by a diagnostic system, wherein the diagnostic system is configured to classify subsequently received treatment profiles using the updated diagnostic configuration.

In some implementations, the non-transitory computer-readable medium can store instructions that cause the at least one processor to receive, via a network interface, an indication identifying one or more misclassified treatment profiles and a corresponding ground-truth label for each misclassified treatment profile. In some implementations, the non-transitory computer-readable medium can store instructions that cause the at least one processor to write the indication and the ground-truth label for each misclassified treatment profile to an audit log data structure stored in the non-transitory memory. In some implementations, the non-transitory computer-readable medium can store instructions that cause the at least one processor to execute the refinement protocol using the audit log data structure to generate the updated diagnostic configuration.

In some implementations, the initial diagnostic configuration comprises a first ruleset comprising a first set of marker values defining a first transition point for classifying a treatment profile into a first disease state and a second ruleset comprising a second set of marker values defining a second transition point for classifying a treatment profile into a second disease state. In some implementations, the first ruleset and the second ruleset are stored as versioned configuration records that are selectable by the diagnostic system at runtime.

In some implementations, the refinement protocol comprises, for each attribute, computing a marker deviation between the first set of marker values and the second set of marker values and storing the marker deviation in a deviation table comprising (i) an attribute identifier, (ii) a biomarker identifier, and (iii) a deviation value. In some implementations, the refinement protocol comprises generating the updated set of marker values by applying the marker deviations from the deviation table to produce an updated ruleset that, when executed by the diagnostic system, reduces a misclassification rate for the plurality of treatment profiles relative to the initial diagnostic configuration.

As described herein, patient data included in treatment profiles can be used to determine an updated set of marker values for a given disease definition that improve diagnostic accuracy for a set of attributes used to diagnose a disease. For example, the system can receive a set of attributes associated with a given disease definition. Each attribute can indicate one or more marker values that are used to diagnose the disease with a first degree of precision. The system can also identify a dataset including treatment profiles associated with individuals who have been diagnosed with the disease. Based on comparing the marker values of the set of attributes with expression levels of biomarkers associated with the attributes in the dataset, the system can determine an updated set of marker values associated with a second degree of precision. The updated set of marker values can be provided to a diagnostic system to configure the diagnostic system to classify subsequent treatment profiles based on the updated set of marker values.

By virtue of determining the updated set of marker values and refining disease definitions as described herein, systems can be configured to mitigate bias introduced by conventional disease definition development. For example, systems can be configured to identify latent patterns across treatment profiles represented as sets of marker values that cannot otherwise be identified through review of these treatment profiles by clinicians or may be otherwise disregarded by clinicians focusing on other sets of marker values believed to be indicative of a given disease. Further, a target degree of precision can be set and the systems described herein can refine a given disease definition to match that target. This can allow for clinicians to target individuals without being over-inclusive or under-inclusive, resulting in extended individual health outcomes. The updated set of marker values can then be provided to a diagnostic system to configure the diagnostic system to classify subsequent treatment profiles based on the updated set of marker values. As opposed to the development of disease definitions using conventional techniques (which can result in disease definitions that are under- or over-inclusive as described), the updated set of marker values refines the scope of the disease definition to, in turn, decrease mistaken diagnoses that can cause unnecessary testing, etc., while allowing for earlier diagnoses of diseases that can improve health outcomes.

### BRIEF DESCRIPTION OF THE DRAWINGS

The accompanying drawings constitute a part of this specification, illustrate one or more embodiments and, together with the specification, explain the subject matter of the disclosure.

FIG. 1 is a block diagram of an environment in which one or more devices operate to process and analyze patient data, in accordance with one or more embodiments described herein.

FIG. 2 is a flow diagram illustrating operations of a method for refining disease definitions, in accordance with one or more embodiments described herein.

FIGS. 3A-3E are a diagram of an example implementation of the method of FIG. 2, in accordance with one or more embodiments described herein.

### DETAILED DESCRIPTION

Reference will now be made to the embodiments illustrated in the drawings, and specific language will be used here to describe the same. It will nevertheless be understood that no limitation of the scope of the disclosure is thereby intended. Alterations and further modifications of the features illustrated here, and additional applications of the principles as illustrated here, which would occur to a person skilled in the relevant art and having possession of this disclosure, are to be considered within the scope of the disclosure.

The system described herein can determine a set of attributes and an associated set of marker values in accordance with a first degree of precision. The system can compare the marker values of biomarkers for each attribute to expression levels of the biomarkers represented by a plurality of treatment profiles of individuals (e.g., that can be patients that were, or are, being treated for a disease) that are diagnosed as having the disease. Based on the comparison, the system can determine a set of updated marker values associated with a second degree of precision and provide data associated with the updated marker values to a diagnostic system to configure the diagnostic system to classify subsequent treatment profiles based on the updated marker values. As a result, the updated set of marker values can more accurately classify treatment profiles. This can improve health outcomes by allowing for quicker identification of individuals having the disease while avoiding unnecessary testing and emotional stress from mistakenly diagnosing individuals with the disease.

The technical advantages of the disclosed systems and methods address fundamental computational challenges in medical diagnostic configuration that cannot be solved through manual review or conventional rules-based approaches. By implementing iterative refinement protocols that process normalized biomarker representations against classification thresholds stored in versioned configuration records, the system mitigates systematic bias introduced when disease definitions are developed based on limited initial datasets or organizational preferences that may not generalize across broader patient populations. The system's use of correlation matrix data structures indexed by attribute identifiers enables automated discovery of biomarker relationships that would be computationally infeasible to identify through manual statistical analysis, particularly when evaluating high-dimensional treatment profile datasets comprising hundreds or thousands of patients with dozens of measured biomarkers per individual. Furthermore, the system's ability to generate time-series records of biomarker expression levels and compute rate-of-change values over defined time windows provides a technical mechanism for encoding dynamic disease progression patterns into diagnostic configurations, transforming static transition-point thresholds into change-pattern thresholds that capture temporal biomarker trajectories indicative of disease onset or advancement. This computational approach addresses diagnostic configuration drift, where initial marker values established during disease definition development become progressively less accurate as new patient data accumulates, by providing an automated feedback mechanism through audit log data structures that capture misclassification events and corresponding ground-truth labels, enabling the refinement protocol to systematically reduce misclassification rates through iterative marker value adjustments that are traceable, reversible via version control, and deployable to distributed diagnostic systems through standardized configuration transmission protocols.

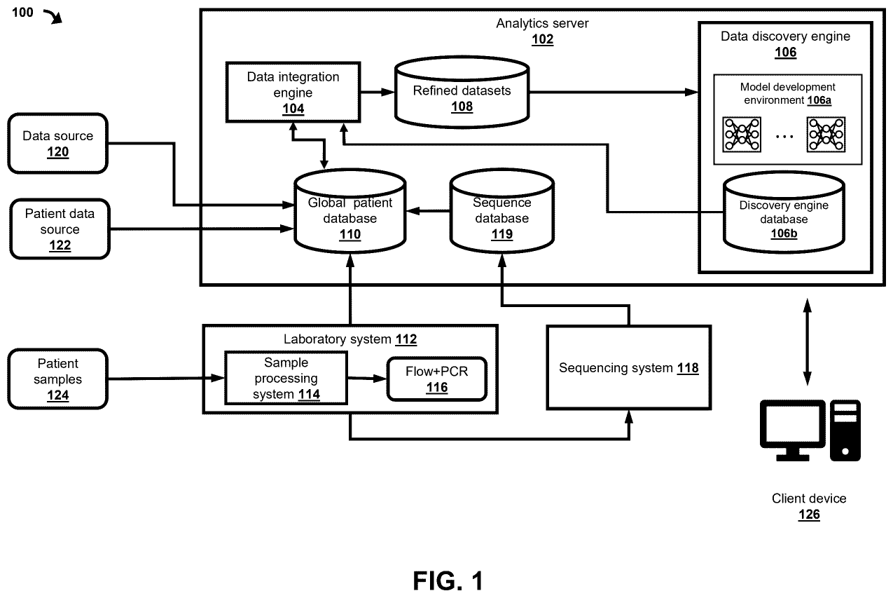

FIG. 1 is a block diagram of a system architecture environment 100 for managing patient data, according to an embodiment. The environment 100 can include an analytics server 102, a laboratory system 112, a sequencing system 118, a data source 120, patient data source 122, patient samples 124, and a client device 126. Various components depicted in FIG. 1 can belong to an organization involved in clinical research of one or more diseases such as, for example, acute myeloid leukemia (AML) or other diseases and/or to one or more organizations involved in treating individuals with the one or more diseases. While certain components and devices are illustrated as being included in the environment 100 of FIG. 1, it will be understood that the environment 100 is not confined to the components or diseases as described herein and can include additional or different components (not shown for purposes of brevity and clarity) which are configured to be considered within the scope of the embodiments described herein.

In some embodiments, the analytics server 102 can include any computing device comprising one or more processors and non-transitory machine-readable storage capable of executing the various tasks, processes, and/or operations as described herein. The analytics server 102 can employ various processors such as central processing units (CPUs), graphical processing units (GPUs), and/or the like. Some non-limiting examples of such computing devices can include workstation computers, laptop computers, server computers, and/or the like. While the environment 100 includes a single analytics server 102, there can be multiple analytics servers 102. Further, the analytics server 102 can include any number of computing devices operating in a distributed computing environment such as, for example, a cloud computing environment. As described herein, the analytics server 102 can include a data integration engine 104, a data discovery engine 106, refined datasets 108, a global patient database 110, and a sequence database 119. In some embodiments, the analytics server 102 can include and/or implement operations that are associated with the laboratory system 112, the sequencing system 118, and/or the client device 126. In some embodiments, the analytics server 102 can include and/or implement operations that are associated with (e.g., involved in the generation of) the data source 120, the patient data source 122, and/or the patient samples 124.

In some embodiments, the analytics server 102 can be configured to receive disease definition data from the data source 120, the patient data source 122, and the laboratory system 112 and sequencing system 118 when processing patient samples 124. For example, the analytics server 102 can be configured to receive disease definition data identifying a plurality of attributes associated with a disease from the data source 120, where each attribute is mapped to at least one biomarker. As an example, as patients interact with clinicians, the clinicians can generate information that is received as input at a client device (not explicitly illustrated) that is associated with the clinicians, the information indicating clinical observations and/or updates to treatment plans for the patients made by the clinician. The client device can then generate patient data that is associated with each patient and representative of the clinical observations or updates to the treatment plans and store the patient data in the data source 120 to later transmit to the analytics server 102. In this example, the analytics server 102 can implement the global patient database 110 such that the patient data is uploaded and stored in the global patient database 110 in association with one or more identifiers for the patient as described herein.

In another example, the analytics server 102 can be configured to receive data from the patient data source 122, where the data is associated with (e.g., represents) information about individual patients. As an example, as a history of a patient is obtained, the clinicians and/or the patients can generate information that is received as input at a client device (not explicitly illustrated) that is associated with the clinicians and/or patients, the information indicating aspects of the history of the patient such as whether the patient is associated with a history of a given disease in their family, whether the patient had any exposure to environmental conditions associated with the given disease, and/or the like. The client device can then generate patient data that is associated with each patient and representative of the history of the patient and store the patient data in the patient data source 122 to later transmit to the analytics server 102. In this example, the analytics server 102 can obtain and store the patient data in the global patient database 110 in association with one or more identifiers for the patient as described herein.

In yet another example, the analytics server 102 can be configured to receive data from the laboratory system 112 and/or the sequencing system 118, where the data is associated with (e.g., represents) information about patient samples (e.g., tissue samples, blood samples, blood counts (e.g., complete blood counts), bone marrow aspiration and biopsy results, lumbar puncture results, and/or the like) as well as the results of the processing of the samples (e.g., a DNA sequence or targets thereof). As an example, as a patient is evaluated and/or treated for a disease such as AML, patient samples 124 similar to those described above can be obtained. The patient samples 124 can be initially obtained and processed by a laboratory system 112 and processed by a sample processing system 114. The sample processing system 114 can implement one or more devices configured to obtain and store the patient samples and extract DNA from the patient samples. For example, in preparation for genetic analysis to guide AML treatment, patient blood or bone marrow can first be obtained from a patient and frozen. Later, these samples can be quality checked to ensure the sample purity and quantity are sufficient for sequencing. In some embodiments, the isolated DNA can then undergo further processing to be separated into manageable fragments and equipped with adapters (e.g., short, specific pieces of synthetic DNA associated with the fragmented DNA molecules) for compatibility with sequencing machines. In some embodiments, the samples can also be provided to a flow and polymerase chain reaction (PCR) system to extract and amplify the isolated DNA. The laboratory system 112 can then provide the processed samples and corresponding data representing the samples to be processed by the sequencing system 118. Additionally, or alternatively, the laboratory system 112 can then provide the data generated by the laboratory system 112 when processing the samples to the analytics server 102 to be stored in the global patient database 110.

In some embodiments, the sequencing system 118 can be configured to receive the patient samples and/or the isolated DNA and sequence the patient samples. In one example, the sequencing system 118 can attach DNA fragments to a surface in a specific pattern, creating clusters. The sequencing itself can involve a series of cycles where fluorescently labeled nucleotides are introduced one by one. The incorporation of each base can be detected, identifying the sequence of the fragment base by base. Finally, the sequencing system 118 can analyze the vast amount of data, assemble the original DNA sequences and identify any variations or mutations present (sometimes referred to as Next-Generation Sequencing (NGS)). The sequencing system 118 can then provide data associated with the sequenced DNA to the analytics server 102. In this example, the analytics server 102 can store the sequenced DNA in a sequence database 119 that stores the sequenced DNA in association with one or more profile identifiers established by the analytics server 102. In some embodiments, the analytics server 102 can also cause the sequence database 119 to provide the data associated with the sequenced DNA to the global patient database 110 to be stored in association with other data associated with the patient such as a treatment profile and/or limited treatment profile for the patient as described herein.

In some embodiments, the analytics server 102 can implement a data integration engine 104 to process data stored in the global patient database 110. For example, the analytics server 102 can implement the data integration engine 104 such that the data integration engine 104 is configured to obtain the data associated with the patients that is stored in the global patient database 110 and processes the data to be used by the data discovery engine 106. In one example, as data is obtained by the global patient database 110 for a given patient, the data can be stored in the global patient database 110 in association with one or more identifiers as part of a profile for the patient. The data integration engine 104 can then obtain the data associated with the patient (e.g., the entire profile or portions thereof) from the global patient database 110 and process the data to generate a limited treatment profile. In some embodiments, for each treatment profile of a plurality of treatment profiles, the data integration engine 104 can generate a normalized biomarker representation comprising quantified expression levels of the biomarkers. The limited treatment profile can then be stored in the refined datasets database 108 (referred to herein as “refined datasets”) and made available to the data discovery engine 106. In this way, the analytics server 102 can maintain two separate datasets that allow for updates to the limited treatment profiles stored in the refined datasets 108 and subsequent use by the data discovery engine 106 when performing the operations described herein. As will be understood, in this example the data associated with the patient that is stored in the global patient database 110 can be updated over time such that the patient profile is represented as a set of entries associated with a time series. As the global patient database 110 is updated, the data integration engine 104 can obtain updated versions of the data associated with the patient from the global patient database 110, process the data when updating the limited treatment profiles in the refined datasets 108, and store the updates in the refined datasets 108.

In some embodiments, the analytics server 102 can implement the data discovery engine 106 that includes a model development environment 106a and a discovery engine database 106b. For example, the analytics server 102 can implement the data discovery engine 106 such that the data discovery engine 106 is configured to receive data associated with one or more limited treatment profiles that are stored in the refined datasets 108 and process the one or more limited treatment profiles. In this example, the analytics server 102 can process the one or more limited treatment profiles using the model development environment 106a. Processing the limited treatment profiles can include providing the limited treatment profiles to one or more models (e.g., machine learning-based models, including supervised models such as linear regression models and unsupervised models such as clustering models, and/or the like) to determine one or more metrics. The one or more metrics can represent the performance of each of the models, indicating which model or groups of models are more or less accurate, efficient, and/or the like at generating one or more predictions compared to one or more other models. These predictions can include indications of treatment options that have a likelihood of optimizing an outcome (e.g., life extension) for the patients. In some implementations, the analytics server 102 can determine an initial diagnostic configuration comprising a set of marker values defining classification thresholds for the plurality of attributes. The analytics server 102 can execute a refinement protocol that iteratively evaluates misclassification of the treatment profiles by the initial diagnostic configuration by comparing the normalized biomarker representation to the classification thresholds. In some implementations, the analytics server 102 can allocate one or more types of processing units to execute the refinement protocol. For example, the analytics server 102 can allocate central processing units (CPUs), graphical processing units (GPUs), field-programmable gate arrays (FPGAs), tensor processing units (TPUs), and/or application-specific integrated circuits (ASICs) to execute the refinement protocol based on the computational requirements of the one or more models. In some implementations, the analytics server 102 can determine the number of each type of processing unit to allocate based on a complexity of the one or more models and/or a size of the limited treatment profiles being processed. For example, the analytics server 102 can increase the number of GPUs allocated when the one or more models include neural network models that perform matrix operations on normalized biomarker representations, and/or can increase the number of CPUs allocated when the one or more models include statistical models that perform sequential comparisons of marker values to quantified expression levels. In some implementations, the analytics server 102 can allocate memory resources to store intermediate results generated during execution of the refinement protocol. For example, the analytics server 102 can allocate random access memory (RAM) to cache the normalized biomarker representations, marker values, and/or classification results determined during iterative evaluation of misclassification. In response to the refinement protocol, the analytics server 102 can generate an updated diagnostic configuration comprising an updated set of marker values that reduce misclassification relative to the initial diagnostic configuration. In some embodiments, the analytics server 102 can process the limited treatment profiles to determine one or more aspects of the limited treatment profiles. For example, where the limited treatment profile is associated with a predetermined number of possible attributes but the patient samples 124 that are available are limited and only usable to determine a subset of the possible attributes, the model development environment 106a can process the portions of the refined patient profile that are available in the refined datasets 108 to determine one or more of the remaining attributes of the possible attributes. In this example, data associated with the one or more remaining attributes can be stored by the data discovery engine 106 in the discovery engine database 106b along with an identifier from the limited treatment profile (e.g., the pseudo-identifier and/or other entries in the limited treatment file). The analytics server 102 can then periodically or in real-time update the global patient database 110 based on the data associated with the limited treatment profiles (e.g., the one or more remaining attributes and/or the like) that are stored in the discovery engine database 106b.

In some embodiments, the data associated with the one or more remaining attributes can be transmitted by the discovery engine database 106b to the data integration engine 104. The data integration engine 104 can use the identifier to determine which treatment profile or limited treatment profile corresponds to the data associated with the one or more remaining attributes. In some embodiments, the data integration engine 104 can then update the treatment profile and/or the limited treatment profile in accordance with the one or more remaining attributes. For example, the data integration engine 104 can update the treatment profile and/or the limited treatment profile with an indication of the appropriate treatment (e.g., that is predicted to optimize the lifespan of the patient) based on analysis of the entries of the treatment profile and/or limited treatment profile. In an example, the data integration engine can access the global patient database 110 and update the entries of the treatment profile in accordance with the remaining attributes. In another example, the data integration engine 104 can access the refined datasets 108 and update the entries of the limited treatment profile in accordance with the remaining attributes.

In some embodiments, the client device 126 can include any computing device comprising a processor and non-transitory machine-readable storage capable of executing the various tasks, processes, and/or operations as described herein. The client device 126 can employ various processors such as central processing units (CPUs), graphical processing units (GPUs), and/or the like. Some non-limiting examples of such computing devices can include workstation computers, laptop computers, server computers, and/or the like. While the environment 100 includes a single client device 126, there can be multiple client devices 126. Further, the client device 126 can include any number of computing devices operating in a distributed computing environment such as, for example, a cloud computing environment. In some embodiments, the client device 126 can be associated with one or more software developers and/or one or more clinicians that are interacting with (e.g., configuring operation of) the analytics server 102 as described herein. In some embodiments, the client device 126 can be associated with one or more clinicians and/or one or more organizations involved in treating patients with the one or more diseases such as a hospital and/or the like. In some embodiments, the analytics server 102 can store the updated diagnostic configuration in a non-transitory memory accessible by the client device 126, wherein the client device 126 is configured to classify subsequently received treatment profiles using the updated diagnostic configuration.

In some embodiments, the analytics server 102 can generate and display an electronic platform (e.g., via the client device 126) when receiving and processing patient data associated with one or more patients, performing one or more operations when analyzing the patient data, and outputting data associated with the results of the operations performed by any of the components of the analytics server 102 such as, for example, the data discovery engine 106. The electronic platform can include graphical user interfaces (GUI) displayed by display devices of one or more client devices 126. An example of the electronic platform generated and hosted by the analytics server 102 can be a web-based application or a website configured to be displayed on different electronic devices, such as mobile devices, tablets, personal computers, and the like.

The above-mentioned components can be configured to interconnect with each other and establish communication connections therebetween through a network (not explicitly illustrated). Examples of the network can include, but are not limited to, private or public local-area-networks (LAN), wireless LAN (WLAN) networks, metropolitan area networks (MAN), wide-area networks (WAN), and the Internet. The network can include wired and/or wireless communications according to one or more standards and/or via one or more transport mediums. The communication over the network can be performed in accordance with various communication protocols such as Transmission Control Protocol and Internet Protocol (TCP/IP), User Datagram Protocol (UDP), and communication protocols. In one example, the network can include wireless communications according to Bluetooth specification sets or another standard or proprietary wireless communication protocol. In another example, the network can also include communications over a cellular network, including, e.g., a GSM (Global System for Mobile Communications), CDMA (Code Division Multiple Access), and EDGE (Enhanced Data for Global Evolution) network.

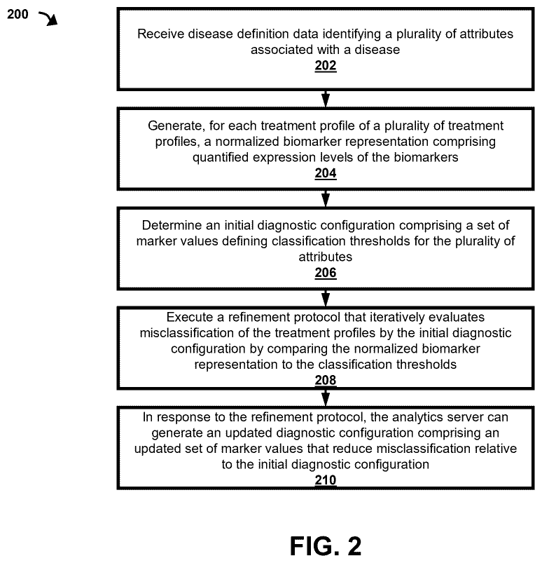

FIG. 2 is a flow diagram of a method 200 for refining disease definitions, in accordance with one or more embodiments described herein. In some implementations, one or more of the functions described with respect to the method 200 can be performed (e.g., completely, partially, and/or the like) by an analytics server (or one or more components thereof) that is the same as, or similar to, the analytics server 102 of FIG. 1. In some implementations, one or more of the functions described with respect to the method 200 can be performed (e.g., completely, partially, and/or the like) by another device or group of devices separate from and/or including the analytics server, such as by one or more client devices that are the same as, or similar to, the client device 126 of FIG. 1.

At operation 202, the analytics server can receive disease definition data identifying a plurality of attributes associated with a disease. For example, the analytics server can receive disease definition data to allow the analytics server to determine a set of attributes represented by the disease definition. The set of attributes can include one or more attributes that are each mapped to one or more biomarkers. In examples, one or more client devices (e.g., that is the same or similar to the client device 126 of FIG. 1) can transmit disease definition data associated with the disease definition to the analytics server to allow the analytics server to determine the set of attributes. These client devices can be associated with (e.g., controlled by) research institutions and/or organizations like the National Comprehensive Cancer Network (NCCN), European LeaukemiaNet (ELN), and/or the like, that are involved in analyzing a given disease and determining attributes and corresponding marker values for a definition of that disease. In another example, the analytics server can retrieve the disease definition data from a database (e.g., that is the same or similar to the discovery engine database 106b of FIG. 1). For example, the analytics server can retrieve the disease definition data from a database maintained by the analytics server when analyzing one or more treatment profiles to refine the disease definition. In these examples, the analytics server can determine or update the marker values for the set of attributes based on analysis and refinement of the disease definition as described herein.

Disease definitions can describe pathological conditions that are identifiable by biological changes that can be assessed through measurement of specific biomarkers of individuals. These biomarkers (represented individually or in various combinations as attributes for a given disease) can encompass cellular, biochemical, or molecular alterations that reflect the presence, severity, or progression of the disease. Types of biomarkers can include diagnostic biomarkers that indicate the presence of the disease, prognostic biomarkers that suggest likely health outcomes or disease progression, predictive biomarkers that help assess responses to specific treatments, etc. The identification and measurement of these biomarkers, and comparison with marker values of one or more disease definitions, can allow for disease diagnosis, progression monitoring, and treatment efficacy evaluation. Thus, within the framework of a given definition, the disease is defined not only by its clinical manifestations but also by its associated measurable biological indicators that provide critical information about the underlying pathological processes associated with the disease.

In some embodiments, each attribute that is indicative of the disease can identify marker values for biomarkers or combinations of biomarkers that, when satisfied, indicate the presence and/or state of progression of the disease in an individual. For example, an attribute can include one or more marker values that represent protein expression of one or more genes or proteins encoded by genes such as, for example, the CD70 gene and/or one or more DNA mutations, indicative of the disease. Additionally or alternatively, an attribute can include one or more biomarkers that can indicate metabolic activity (e.g., glucose, cholesterol, and/or the like) or physiological measurements (e.g., blood pressure, heart rate, and/or the like) or other types of biomarkers (e.g., non-specific biomarkers of inflammation such as white blood cell (WBC) count, etc.) that can be indicative of the disease. It will be understood that the attributes can be associated with any suitable biomarker or combinations of biomarkers other than those explicitly described herein.

In some embodiments, the disease definition data can include a first set of attributes associated with a first organization and a second set of attributes associated with a second organization as described herein. For example, different organizations can establish respective disease definitions with sets of attributes for diagnosis of the disease. In these examples, the attributes for the disease definitions of two or more organizations can establish different combinations of biomarkers and/or marker values. For example, each of the two or more organizations can determine a set of attributes having marker values for biomarkers identified by the attribute that can vary to indicate whether individuals with expression levels for corresponding biomarkers that satisfy the attribute set by the respective organizations have the disease (e.g., at one or more stages of the disease). The analytics server can then be configured to analyze the various sets of marker values to determine respective degrees of precision for the marker values established by each organization's definition for the disease and select a definition and/or refine a definition as described herein.

In embodiments, biomarkers can be correlated with one another to indicate the state of the individual. For example, expression levels for several biomarkers can be obtained to indicate the state of the individual at one or more points in time. In the examples described herein, when each expression level represented by a treatment profile of an individual satisfies the marker values set by an attribute included in the disease definition of a particular organization, the individual can be identified as having the disease and/or the progression of that disease from state to state. In other examples, the state of the individual can indicate whether the individual has disease (e.g., AML) and/or a subtypes for that disease (e.g., French-American-British (FAB) classification of AML, World Health Organization classification of AML, and/or the like).

At operation 204, the analytics server can generate, for each treatment profile of a plurality of treatment profiles, a normalized biomarker representation comprising quantified expression levels of the biomarkers. For example, the analytics server can obtain treatment profiles stored in a global patient database (e.g., that is the same as, or similar to, the global patient database 110 of FIG. 1) and/or limited treatment profiles stored in refined datasets (e.g., that is the same as, or similar to, the refined datasets 108 of FIG. 1). The analytics server can process each treatment profile to generate a normalized biomarker representation that includes quantified expression levels of the biomarkers mapped to the attributes in the disease definition data. In some embodiments, generating the normalized biomarker representation can include standardizing measurements of biomarker expression levels to allow for consistent comparison across treatment profiles. For example, the analytics server can normalize expression levels by applying scaling factors, converting raw measurements to standardized units, and/or computing relative expression values. The normalized biomarker representation can then be used by the analytics server when determining and refining marker values for the disease definition as described herein.

At operation 206, the analytics server can determine an initial diagnostic configuration comprising a set of marker values defining classification thresholds for the plurality of attributes. For example, the analytics server can determine a set of marker values for the attributes that are established by the disease definition as being indicative of the disease. Each marker value of the set of marker values for an attribute (alone or in combination with other marker values) can establish a classification threshold or combination of classification thresholds which, when satisfied by expression levels of a given individual, indicate presence of the disease. For example, a quantified expression level of a biomarker as measured in an individual that satisfies (e.g., that is at or above) the associated marker value for an attribute of a disease definition can indicate presence of the disease. Additionally, or alternatively, a quantified expression level of a biomarker as measured in an individual that satisfies (e.g., is below) the associated marker value can indicate presence of the disease. Where the quantified expression levels as measured in an individual (e.g., represented by a normalized biomarker representation) satisfy corresponding marker values set by an attribute of the disease definition, the analytics server can determine that the treatment profile represents an individual with the disease.

In some embodiments, to define the disease in terms of its progression, a given attribute of a definition can include a first set of marker values that establish a first classification threshold indicating presence of the disease in a first state and a second set of marker values that establish a second classification threshold indicating presence of the disease in a second state. The first state can indicate presence of the disease with a lesser degree of severity (e.g., lower-risk AML or early-detected AML, and/or the like) and the second state of the disease can indicate presence of the disease with a greater degree of severity. In another example, the first state can be a first type (e.g., FAB MI AML, AML with maturations, Acute Promyelocytic Leukemia (APL), and/or the like) of the disease and the second state can be a second type of the disease that represents a progression of the disease. As described herein, quantified expression levels of biomarkers measured for an individual that satisfy the first set of marker values or the second set of marker values can indicate whether the disease is present in the individual, as well as a state of progression for that disease. In some implementations, the analytics server can store the first set of marker values as a first ruleset and the second set of marker values as a second ruleset, where the first ruleset and the second ruleset are stored as versioned configuration records that are selectable by a diagnostic system at runtime. For simplicity, the present disclosure will be discussed with respect to refining a single set of marker values for a definition. However, it will be understood that the analytics server can refine multiple sets of marker values used to diagnose individuals as a disease progresses.

In some embodiments, when evaluating disease definition data from multiple organizations, the analytics server can generate the initial diagnostic configuration by comparing a first set of marker values associated with a first set of attributes from a first organization with a second set of marker values associated with a second set of attributes from a second organization. For example, to evaluate multiple, deviating definitions from organizations for the disease, the analytics server can determine multiple sets of marker values that correspond to organizations that are developing definitions for the disease. This can include a first set of marker values (e.g., corresponding to the disease definition from a first organization) and a second set of marker values (e.g., corresponding to the disease definition from a second organization). The analytics server can then analyze the various sets of marker values to determine respective degrees of precision for the marker values established by each organization's definition for the disease and select marker values for the initial diagnostic configuration and/or refine the initial diagnostic configuration as described herein. In some implementations, the analytics server can generate the initial diagnostic configuration by producing a merged configuration record that (i) maps each attribute to a selected marker value chosen from the first set of marker values or the second set of marker values and (ii) stores, for each selected marker value, provenance metadata identifying the organization from which the selected marker value originated. While the present disclosure is described with respect to iterative refinement of a single disease definition, it will be understood that at least some of the operations described herein can be implemented to allow the analytics server to select a disease definition from among a plurality of disease definitions and/or to iteratively refine multiple disease definitions.

At operation 208, the analytics server can execute a refinement protocol that iteratively evaluates misclassification of the treatment profiles by the initial diagnostic configuration by comparing the normalized biomarker representation to the classification thresholds. For example, a system (e.g., that is the same or similar to the data discovery engine 106 of FIG. 1) implemented by the analytics server can compare the marker values of the initial diagnostic configuration to the quantified expression levels (e.g., marker values measured for a particular individual) represented by the normalized biomarker representations of treatment profiles and/or limited treatment profiles. In this example, the comparison can be both to treatment profiles for individuals that are known to have the disease and/or to treatment profiles for individuals that are known to not have the disease. The refinement protocol can iteratively evaluate whether the initial diagnostic configuration correctly or incorrectly classifies treatment profiles. For example, the analytics server can access a test set of treatment profiles that are identified (e.g., tagged) as being associated with individuals having the disease or not having the disease. The test set of treatment profiles can include one or more treatment profiles stored in a global patient database (e.g., that is the same as, or similar to, the global patient database 110 of FIG. 1) and/or the one or more limited treatment profiles stored in refined datasets (e.g., that is the same as, or similar to, the refined datasets 108 of FIG. 1). The analytics server can then classify the test set of treatment profiles according to the initial diagnostic configuration and compare the classifications to the labels to evaluate misclassification.

In some embodiments, the analytics server can execute the refinement protocol in response to receiving an indication that the initial diagnostic configuration incorrectly classifies one or more treatment profiles. For example, the analytics server can receive, via a network interface, an indication identifying one or more misclassified treatment profiles and a corresponding ground-truth label for each misclassified treatment profile. The analytics server can write the indication and the ground-truth label for each misclassified treatment profile to an audit log data structure stored in non-transitory memory. The analytics server can then execute the refinement protocol using the audit log data structure to generate the updated diagnostic configuration. This indication can be received in response to the identification of one or more misclassifications of the treatment profiles by a client device or one or more systems implemented by the analytics server. In one example, the client device can submit the indication to the analytics server in response to the client device determining that one or more treatment profiles have been misclassified along with a request to refine the definition used by the client device.

When evaluating the initial diagnostic configuration through the refinement protocol, the analytics server can determine that the set of marker values for the definition are or are not satisfied by one or more treatment profiles of individuals known to have the disease. Additionally, or alternatively, the analytics server can determine that the set of marker values for the definition are or are not satisfied by one or more treatment profiles of individuals known to not have the disease. The analytics server can then compare the number of treatment profiles that were correctly identified to the number of treatment profiles that were not correctly identified and evaluate the degree of misclassification in response to this comparison. This refinement protocol can be iteratively performed as the analytics server evaluates the initial diagnostic configuration and generates updated marker values when targeting a desired reduction in misclassification.

At operation 210, in response to the refinement protocol, the analytics server can generate an updated diagnostic configuration comprising an updated set of marker values that reduce misclassification relative to the initial diagnostic configuration. For example, the analytics server can determine a set of updated marker values that reduce misclassification of treatment profiles when compared to the initial diagnostic configuration. In examples, where a narrower definition is desired, an updated marker value in the set of updated marker values can be set by the analytics server as being higher than the original marker value and can result in a comparatively higher threshold for indicating presence of the disease. In this example, the updated set of marker values can narrow the disease definition so as to reduce false positives that can lead to undesirable additional testing of individuals that do not have the disease. In another example, where a broader definition is desired, the updated marker value in the set of updated marker values can be set by the analytics server lower than the original marker value and can result in a comparatively lower threshold for indicating presence of the disease. In this example, the updated set of marker values can broaden the disease definition so as to reduce false negatives that can lead to underdiagnosed individuals, particularly for individuals who have earlier forms or stages of the disease.

In some embodiments, the analytics server can update the set of attributes to include or exclude marker values for certain biomarkers based on determining correlations between one or more attributes and the disease. For example, the analytics server can obtain access to a set of treatment profiles associated with individuals as described herein. The analytics server can then determine correlations between a subset of the attributes across the set of treatment profiles that are associated with individuals known to have the disease by generating a correlation matrix data structure storing correlation coefficients indexed by attribute identifiers. These attributes may or may not be included in the disease definition being refined. In an example, the correlations can indicate that at least one different attribute (e.g., a different biomarker or combination of biomarkers) is not included in the plurality of attributes indicative of the disease but nonetheless are correlated with the presence or absence of the disease. The analytics server can identify the at least one different attribute based on correlation coefficients in the correlation matrix satisfying a selection criterion stored in memory. When updating the plurality of attributes, the analytics server can then include the at least one different attribute and/or remove at least one attribute that is determined not to be correlated with the disease by updating an attribute registry that defines which biomarkers are ingested into the normalized biomarker representation. As a result, the analytics server can identify attributes (e.g., biomarkers or sets of biomarkers) that are not included and/or are different from those in the disease definition and update the disease definition to include these attributes. In this way, the analytics server can identify attributes that are more readily available, less invasive, and/or less expensive than one or more other attributes when updating the plurality of attributes to achieve a targeted reduction in misclassification. In response to updating the plurality of attributes by updating the attribute registry, the analytics server can regenerate the initial diagnostic configuration by generating a new versioned configuration record that includes marker values for the at least one different attribute.

In some embodiments, the refinement protocol can include determining, for each attribute, a marker deviation based on a first set of marker values and a second set of marker values, and generating the updated set of marker values based on the marker deviation. For example, when iteratively updating the marker values for an attribute of a given disease definition, the analytics server can determine the set of updated marker values based on marker deviations between two or more earlier-analyzed sets of marker values. In one example, where the set of marker values includes a first set of marker values evaluated at a first point in time and a second set of marker values evaluated at a second point in time, the analytics server can compute a marker deviation between the first and second set of marker values and store the marker deviation in a deviation table comprising (i) an attribute identifier, (ii) a biomarker identifier, and (iii) a deviation value. The analytics server can then generate the updated set of marker values by applying the marker deviations from the deviation table to produce an updated ruleset that, when executed by a diagnostic system, reduces a misclassification rate for the plurality of treatment profiles relative to the initial diagnostic configuration. For example, where the deviation indicates that the iterative refinement of the definition is improving the reduction in misclassification for a given attribute, the analytics server can determine the set of updated marker values to similarly extend the deviation.

In some embodiments, the refinement protocol can include comparing each marker deviation to a corresponding expression level of attributes across the plurality of treatment profiles, and generating the updated set of marker values in response to comparing each marker deviation to corresponding expression levels such that the updated diagnostic configuration satisfies a target precision threshold. For example, the analytics server can determine a data trend (mean, median, standard deviation, and/or the like) from the quantified expression levels of biomarkers represented by the normalized biomarker representations of treatment profiles. In some examples, the refinement protocol can include generating, for each treatment profile, a normalized biomarker feature vector comprising normalized expression levels corresponding to the attributes in the merged configuration record. The marker deviation can be compared to the normalized biomarker feature vectors across the plurality of treatment profiles to compute a precision metric for the merged configuration record. In an embodiment, the analytics server can generate the updated set of marker values by modifying the merged configuration record until the precision metric satisfies a target precision threshold, and storing the updated diagnostic configuration as a new version of the merged configuration record. In examples, the set of updated marker values can satisfy a target precision threshold which is determined by the analytics server when iteratively refining the disease definition through the refinement protocol.

In some embodiments, the refinement protocol can include determining a change pattern indicative of changes in the marker values for each attribute over time, wherein the change pattern indicates changes in the associated attribute that are indicative of the disease, and generating the updated set of marker values based on the change pattern. For example, the analytics server can determine the set of updated marker values based on change patterns representing changes in expression levels measured in individuals over time. In an example, the analytics server can determine a change pattern indicative of changes in marker values for each attribute of a definition by generating a time-series record of biomarker expression levels for at least a subset of the treatment profiles. The analytics server can determine the change pattern by computing, from the time-series record, at least one rate-of-change value for the biomarker expression level over a defined time window. The analytics server can then determine that a sudden change in an expression level of a biomarker (e.g., increase, decrease, and/or the like) that satisfies the changes in marker values is indicative of the disease when measured in an individual. In some examples, the analytics server can then generate the updated set of marker values based on the rate-of-change value such that the updated diagnostic configuration classifies subsequent treatment profiles using both (i) a transition-point marker value and (ii) a change-pattern threshold derived from the rate-of-change value. This set of updated marker values can denote the change in expression levels of corresponding biomarkers over a period of time as opposed to a single classification threshold value.

The analytics server can store the updated diagnostic configuration in a non-transitory memory accessible by a diagnostic system, wherein the diagnostic system is configured to classify subsequently received treatment profiles using the updated diagnostic configuration. For example, the diagnostic system can be configured to receive a treatment profile identifying a set of quantified expression levels of biomarkers measured in an individual. The diagnostic system can then be configured to determine whether the individual has the disease based on a comparison of the updated marker values to the quantified expression levels of the biomarkers as measured in the individual. In an example, the diagnostic system can be a client device that provided the disease definition data. In some examples, the diagnostic system can be associated with a clinician diagnosing individuals as having or not having the disease. The diagnostic system can be configured to classify subsequent treatment profiles based on the updated marker values in response to receiving the updated diagnostic configuration. For example, the diagnostic system can be configured to determine whether a received treatment profile has the disease. In this example, the diagnostic system can be a rules-based system that classifies the received treatment profile based on comparing the biomarker expression levels to the updated set of marker values. In some examples, the diagnostic system can classify the received treatment profile as being diagnosed with the disease in response to determining that one or more biomarker expression levels measured in the individual satisfies a threshold set by one or more marker values in the set of updated marker values. As will be understood, the treatment profiles used to refine a given disease definition can include treatment profiles stored in a global patient database and/or limited treatment profiles stored in refined datasets. For example, each treatment profile can have a corresponding limited treatment profile that alters (e.g., removes, updates, etc.) information that identifies the individual corresponding to the treatment profile. In this example, one or more pieces of identifiable information (e.g., name, birthdate, and/or the like) can be removed or updated in the treatment profile to create the corresponding limited treatment profile.

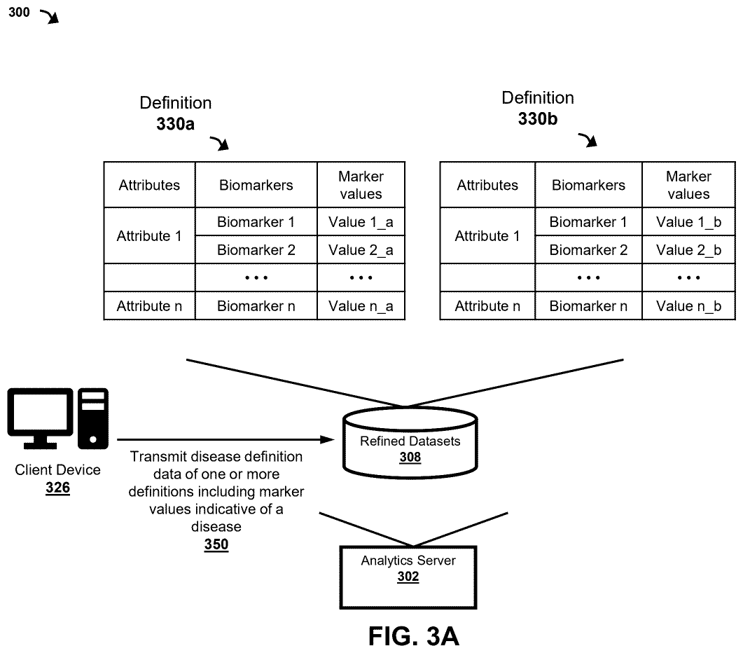

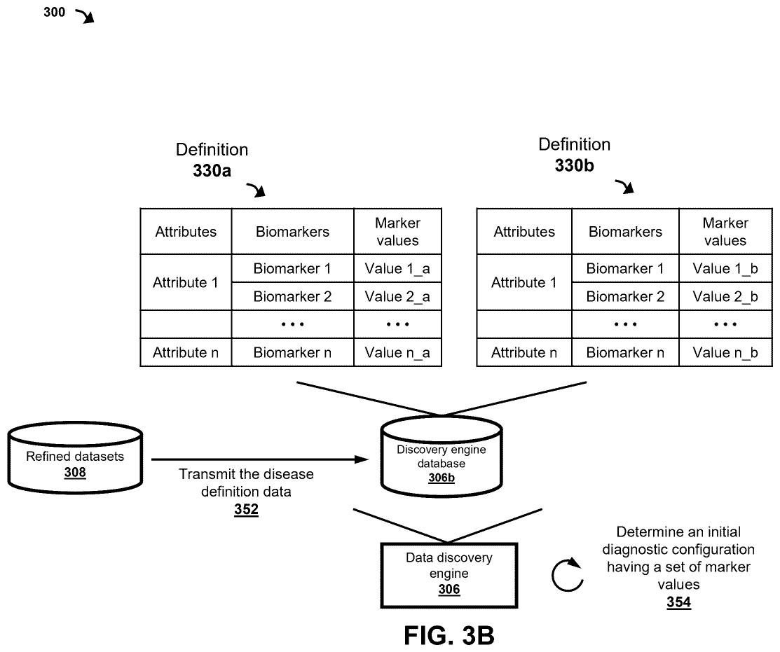

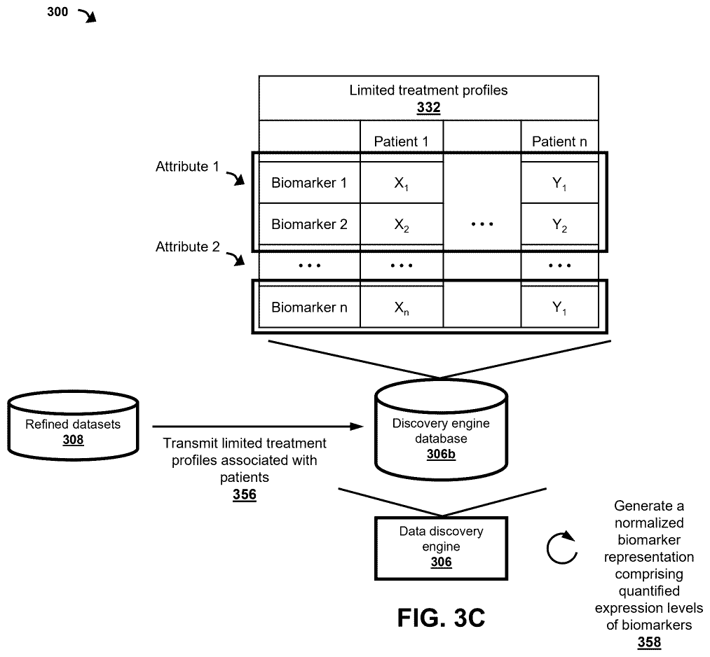

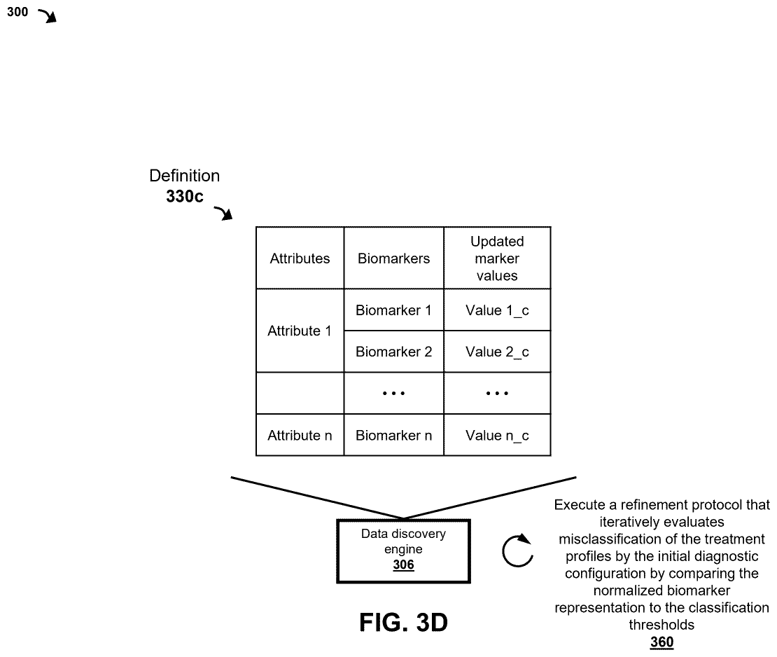

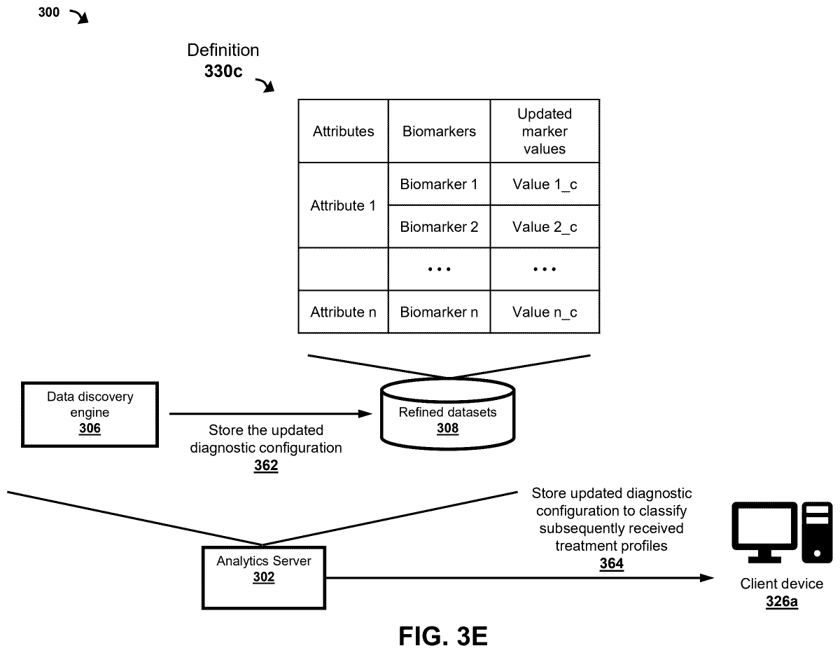

FIGS. 3A-3E are a diagram of an example implementation 300 of the method 200 of FIG. 2, in accordance with one or more embodiments described herein. In some embodiments, the operations of the implementation 300 can be implemented by a client device 326, an analytics server 302, and refined datasets 308, that are the same as, or similar to, the client device 126, analytics server 102, and the refined datasets 108 of FIG. 1. Additionally, or alternatively, one or more of the operations of the implementation 300 can involve a data discovery engine 306 and a discovery engine database 306b that are the same as, or similar to, data discovery engine 106 and a discovery engine database 106b of FIG. 1.

At operation 350, disease definition data of one or more disease definitions (e.g., 330a, 330b) can be transmitted by a client device 326 to the analytics server 302. In an embodiment, the analytics server 302 can store the disease definition data in refined datasets 308 based on receipt of the disease definition data. The disease definition data can identify a plurality of attributes (e.g., Attribute 1, . . . . Attribute n) associated with a disease, where each attribute is mapped to at least one biomarker (e.g., Biomarker 1, Biomarker 2, . . . . Biomarker n). In some embodiments, the disease definition data can include marker values (e.g., Value 1_a, Value 2_a, . . . . Value n_a) defining classification thresholds at which the attributes are indicative of the disease. In examples, each attribute can be mapped to one or more biomarkers. In some examples, a biomarker can be mapped to more than one attribute and can have a different marker value at each occurrence.

At operation 352, the analytics server 302 can provide the disease definition data associated with one or more disease definitions to the data discovery engine 306. In an example, the analytics server 302 can cause the disease definition data to be provided to the data discovery engine 306 in response to receiving the disease definition data. In an embodiment, the data discovery engine 306 can store the disease definition data in the discovery engine database 306b. For example, the data discovery engine 306 can store the disease definition data in the discovery engine database 306b in response to receiving the disease definition data. As will be understood, the data discovery engine 306 can be configured to perform some or all of the operations described with respect to the analytics server of FIGS. 1 and 2.

At operation 354, the data discovery engine 306 can determine an initial diagnostic configuration comprising a set of marker values (e.g., Value 1_a, Value 2_a, . . . . Value n_a) defining classification thresholds for the plurality of attributes. For example, the data discovery engine 306 can determine the initial diagnostic configuration based on receiving the disease definition data. In an example, the marker values can establish classification thresholds at which the associated biomarker indicates presence of a disease. For example, a biomarker can indicate presence of a disease when its quantified expression level is at or above a classification threshold given by a marker value. In some embodiments, the initial diagnostic configuration can comprise a first set of marker values establishing a first classification threshold indicating presence of the disease in a first state and a second set of marker values establishing a second classification threshold indicating presence of the disease in a second state. For example, the first and second set of marker values can represent a first severity of the disease and a second (e.g., greater) severity of the disease. In another example, the first and second set of marker values can represent different types of the disease. In some embodiments, disease definition data from two or more organizations can be transmitted to the data discovery engine. For example, the disease definition data can be two disease definitions for the diagnosis of a disease that have been established by two different medical organizations. In this example, the data discovery engine can generate the initial diagnostic configuration by comparing a first set of marker values associated with a first set of attributes with a second set of marker values associated with a second set of attributes. For example, the data discovery engine can determine, for each attribute, whether the initial diagnostic configuration should include a marker value from the first set of marker values or the second set of marker values.

At operation 356, the analytics server 302 can provide limited treatment profiles 332 associated with individuals to the data discovery engine 306. For example, the analytics server 302 can cause limited treatment profiles 332 to be transmitted from the refined datasets 308 to the discovery engine database 306b. In some examples, the analytics server can transmit the limited treatment profiles 332 to the data discovery engine 306 based on transmitting the disease definition data to the data discovery engine 306. In some embodiments, the set of limited treatment profiles 332 can include limited treatment profiles associated with individuals who have been diagnosed with the disease associated with the disease definition data. For example, the analytics server 302 can identify the disease associated with the disease definition data and transmit limited treatment profiles 332 associated with individuals diagnosed as having the disease to the data discovery engine based on receiving the disease definition data. In other examples, the limited treatment profiles 332 can be associated with individuals that are diagnosed as not having the disease.

At operation 358, the data discovery engine 306 can generate, for each treatment profile of the limited treatment profiles 332, a normalized biomarker representation comprising quantified expression levels (e.g., X₁, X₂, . . . . Xₙ) of biomarkers. For example, the data discovery engine 306 can generate the normalized biomarker representations based on receiving the set of limited treatment profiles 332. In some examples, a quantified expression level can be a quantitative measurement related to the biomarker. In an example wherein one of the biomarkers is a membrane protein (e.g., CD70, CD33, and/or the like), the quantified expression level can be the percentage of cells in a sample that express the membrane protein. In some embodiments, generating the normalized biomarker representation can include standardizing measurements of biomarker expression levels to allow for consistent comparison across treatment profiles.

At operation 360, the data discovery engine 306 can execute a refinement protocol that iteratively evaluates misclassification of the treatment profiles by the initial diagnostic configuration by comparing the normalized biomarker representation to the classification thresholds. In some examples, the data discovery engine 306 can include a model (e.g., statistical model, artificial intelligence model, and/or the like). The data discovery engine 306 can use the model when executing the refinement protocol. For example, the model can iteratively evaluate misclassification based on receiving the normalized biomarker representations of the treatment profiles. In an example, the model can be a statistical model that compares the quantified expression levels of biomarkers represented by the normalized biomarker representations to the marker values of the initial diagnostic configuration. In another example, the model can be an artificial intelligence model that evaluates misclassification based on receiving the normalized biomarker representations and/or the initial diagnostic configuration as input.

In response to the refinement protocol, the data discovery engine 306 can generate an updated diagnostic configuration 330c comprising an updated set of marker values that reduce misclassification relative to the initial diagnostic configuration. In some embodiments, the data discovery engine 306 can execute the refinement protocol in response to receiving an indication that the initial diagnostic configuration incorrectly classifies one or more treatment profiles. For example, the client device 326a can submit an indication that the initial diagnostic configuration incorrectly classified one or more treatment profiles. In an example, the data discovery engine 306 can execute the refinement protocol in response to receiving this indication. In other embodiments, the refinement protocol can include determining a change pattern indicative of changes in the marker values for each attribute over time. In these embodiments, the data discovery engine 306 can determine a change pattern indicative of changes in the marker values for one or more attributes in the plurality of attributes. In some examples, the change pattern can indicate changes in the associated attribute over time that are indicative of the disease. For example, the data discovery engine 306 can determine that a sharp increase in a quantified expression level of a certain biomarker is indicative of the disease. In some examples, the data discovery engine 306 can then generate the updated set of marker values based on the change pattern. In other embodiments, the data discovery engine 306 can generate the updated set of marker values in response to determining correlations between a subset of the attributes across the plurality of treatment profiles. In some examples, the correlations can indicate that at least one different attribute is not included in the plurality of attributes indicative of the disease. For example, a first attribute can be removed in response to determining that it is correlated with a second attribute that is cheaper to test for. The data discovery engine 306 can update the plurality of attributes to include the at least one different attribute and regenerate the initial diagnostic configuration in response to updating the plurality of attributes. In other embodiments wherein the initial diagnostic configuration includes a first set of marker values indicating presence of the disease in a first state and a second set of marker values indicating presence of the disease in a second state, the refinement protocol can include determining, for each attribute, a marker deviation based on the first set of marker values and the second set of marker values, and generating the updated set of marker values based on the marker deviation. In an example, the marker deviation can be the difference between a marker value in the first set of marker values and a marker value in the second set of marker values that are associated with the same attribute. In some examples, the refinement protocol can include comparing each marker deviation to a corresponding quantified expression level of attributes across the plurality of treatment profiles, and generating the updated set of marker values in response to comparing each marker deviation to corresponding quantified expression levels such that the updated diagnostic configuration satisfies a target precision threshold.

At operation 362, the analytics server 302 can store the updated diagnostic configuration 330c in the refined datasets 308. In some examples, the analytics server 302 can cause the data discovery engine 306 to provide the updated diagnostic configuration 330c comprising the updated marker values to the refined datasets 308. In an example, the refined datasets 308 can update an existing disease definition with the updated marker values.

At operation 364, the analytics server 302 can store the updated diagnostic configuration 330c in a non-transitory memory accessible by the client device 326a. In some examples, the client device 326a can be the client device 326. In other examples, the client device 326a can be a separate diagnostic system. In examples, the client device 326a can be configured to classify subsequently received treatment profiles using the updated diagnostic configuration in response to receiving the updated diagnostic configuration. For example, the client device 326a can be a rules-based system that can be configured to diagnose an individual in response to determining that a pre-determined number of attributes from a treatment profile associated with the individual meet the classification thresholds set by the updated marker values.

The various illustrative logical blocks, modules, circuits, and algorithm steps described in connection with the embodiments disclosed herein can be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans can implement the described functionality in varying ways for each particular application, but such implementation decisions should not be interpreted as causing a departure from the scope of this disclosure or the claims.

Some embodiments of the present disclosure are described herein in connection with a threshold. As described herein, satisfying a threshold may refer to a value being greater than the threshold, more than the threshold, higher than the threshold, greater than or equal to the threshold, less than the threshold, fewer than the threshold, lower than the threshold, less than or equal to the threshold, equal to the threshold, and/or the like.

Embodiments implemented in computer software can be implemented in software, firmware, middleware, microcode, hardware description languages, or any combination thereof. A code segment or machine-executable instructions can represent a procedure, a function, a subprogram, a program, a routine, a subroutine, a module, a software package, a class, or any combination of instructions, data structures, or program statements. A code segment can be coupled to another code segment or a hardware circuit by passing and/or receiving information, data, arguments, parameters, or memory contents. Information, arguments, parameters, data, etc., can be passed, forwarded, or transmitted via any suitable means, including memory sharing, message passing, token passing, network transmission, etc.

The actual software code or specialized control hardware used to implement these systems and methods is not limiting of the claimed features or this disclosure. Thus, the operation and behavior of the systems and methods were described without reference to the specific software code being understood that software and control hardware can be designed to implement the systems and methods based on the description herein.

When implemented in software, the functions can be stored as one or more instructions or code on a non-transitory computer-readable or processor-readable storage medium. The steps of a method or algorithm disclosed herein can be embodied in a processor-executable software module, which can reside on a computer-readable or processor-readable storage medium. A non-transitory computer-readable or processor-readable media includes both computer storage media and tangible storage media that facilitate the transfer of a computer program from one place to another. A non-transitory processor-readable storage media can be any available media that can be accessed by a computer. By way of example, and not limitation, such non-transitory processor-readable media can comprise RAM, ROM, EEPROM, CD-ROM or other optical disk storage, magnetic disk storage or other magnetic storage devices, or any other tangible storage medium that can be used to store desired program code in the form of instructions or data structures and that can be accessed by a computer or processor. Disk and disc, as used herein, include compact disc (CD), laser disc, optical disc, digital versatile disc (DVD), floppy disk, and Blu-ray disc where disks usually reproduce data magnetically, while discs reproduce data optically with lasers. Combinations of the above should also be included within the scope of computer-readable media. Additionally, the operations of a method or algorithm can reside as one or any combination or set of codes and/or instructions on a non-transitory processor-readable medium and/or computer-readable medium, which can be incorporated into a computer program product.

The preceding description of the disclosed embodiments is provided to enable any person skilled in the art to make or use the embodiments described herein and variations thereof. Various modifications to these embodiments will be readily apparent to those skilled in the art, and the principles defined herein can be applied to other embodiments without departing from the spirit or scope of the subject matter disclosed herein. Thus, the present disclosure is not intended to be limited to the embodiments shown herein but is to be accorded the widest scope consistent with the following claims and the principles and novel features disclosed herein.

While various aspects and embodiments have been disclosed, other aspects and embodiments are contemplated. The various aspects and embodiments disclosed are for purposes of illustration and are not intended to be limiting, with the true scope and spirit being indicated by the following claims.

What is claimed is:

1. A system for refining criteria associated with a disease to improve disease diagnosis at earlier stages of disease progression, the system comprising:
one or more processors configured to:
receive disease definition data identifying a plurality of attributes associated with a disease, each attribute mapped to at least one biomarker;
generate, for each treatment profile of a plurality of treatment profiles, a normalized biomarker representation comprising quantified expression levels of the biomarkers;
determine an initial diagnostic configuration comprising a set of market values defining classification thresholds for the plurality of attributes;
execute a refinement protocol that iteratively evaluates misclassification of the treatment profiles by the initial diagnostic configuration by comparing the normalized biomarker representation to the classification thresholds;
in response to the refinement protocol, generate an updated diagnostic configuration comprising an updated set of market values that reduce misclassification relative to the initial diagnostic configuration; and
store the updated diagnostic configuration in a non-transitory memory accessible by the diagnostic system, wherein the diagnostic system is configured to classify subsequently received treatment profiles using the updated diagnostic configuration.

2. The system of claim 1, wherein the one or more processors are further configured to:
receive, via a network interface, an indication identifying one or more classified treatment profiles and a corresponding ground-truth label for each misclassified treatment profile;
write the indication, the ground-truth label for each misclassified treatment profile; and
execute the refinement protocol using the audit log data structure to generate the updated diagnostic configuration.

3. The system of claim 1, wherein the initial diagnostic configuration comprises:
a first ruleset comprising a first set of marker values defining a first transition point for classifying a treatment profile into a first disease state; and
a second ruleset comprising a second set of marker values defining a second transition point for classifying a treatment profile into a second disease state,
wherein the first ruleset and the second ruleset are stored as versioned configuration records that are selectable by the diagnostic system at runtime.

4. The system of claim 3, wherein the refinement protocol comprises:
for each attribute, computing a marker deviation between the first set of marker values and the second set of marker values and storing the marker deviation in a deviation table comprising (i) an attribute identifier, (ii) a biomarker identifier, and (iii) a deviation value; and
generating the updated set of marker values by applying the marker deviations from the deviation table to produce an updated ruleset that, when executed by the diagnostic system, reduces a misclassification rate for the plurality of treatment profiles relative to the initial diagnostic configuration.

5. The system of claim 1, wherein the disease definition data comprises a first set of attributes associated with a first organization and a second set of attributes associated with a second organization; and
wherein the one or more processors are configured to:
generate the initial diagnostic configuration by producing a merged configuration record that (i) maps each attribute to a selected marker value chosen from the first set of marker values or the second set of marker values and (ii) stores, for each selected marker value, provenance metadata identifying the organization from which the selected marker value originated.

6. The system of claim 5, wherein the refinement protocol comprises:
generating, for each treatment profile, a normalized biomarker feature vector comprising normalized expression levels corresponding to the attributes in the merged configuration record;
comparing each marker deviation to the normalized biomarker feature vectors across the plurality of treatment profiles to compute a precision metric for the merged configuration record; and
generating the updated set of marker values by modifying the merged configuration record until the precision metric satisfies a target precision threshold, and storing the updated diagnostic configuration as a new version of the merged configuration record.

7. The system of claim 1, wherein the refinement protocol comprises:
for each attribute, generating a time-series record of biomarker expression levels for at least a subset of the treatment profiles;
determining the change pattern by computing, from the time-series record, at least one rate-of-change value for the biomarker expression level over a defined time window; and
generating the updated set of marker values based on the rate-of-change value such that the updated diagnostic configuration classifies subsequent treatment profiles using both (i) a transition-point marker value and (ii) a change-pattern threshold derived from the rate-of-change value.

8. The system of claim 1, wherein the one or more processors are further configured to:
determine correlations between a subset of the attributes across the plurality of treatment profiles by generating a correlation matrix data structure storing correlation coefficients indexed by attribute identifiers;
identify the at least one different attribute based on correlation coefficients in the correlation matrix satisfying a selection criterion stored in memory;
update the plurality of attributes by updating an attribute registry that defines which biomarkers are ingested into the normalized biomarker representation; and
in response to updating the attribute registry, regenerate the initial diagnostic configuration by generating a new versioned configuration record that includes marker values for the at least one different attribute.

9. A method for refining criteria associated with a disease to improve disease diagnosis at earlier stages of disease progression, the method comprising:
receiving, by one or more processors, disease definition data identifying a plurality of attributes associated with a disease, each attribute mapped to at least one biomarker;
generating, by the one or more processors, for each treatment profile of a plurality of treatment profiles, a normalized biomarker representation comprising quantified expression levels of the biomarkers;
determining, by the one or more processors, an initial diagnostic configuration comprising a set of marker values defining classification thresholds for the plurality of attributes;
executing, by the one or more processors, a refinement protocol that iteratively evaluates misclassification of the treatment profiles by the initial diagnostic configuration by comparing the normalized biomarker representation to the classification thresholds;
in response to the refinement protocol, generating, by the one or more processors, an updated diagnostic configuration comprising an updated set of marker values that reduce misclassification relative to the initial diagnostic configuration; and
storing, by the one or more processors, the updated diagnostic configuration in a non-transitory memory accessible by a diagnostic system, wherein the diagnostic system is configured to classify subsequently received treatment profiles using the updated diagnostic configuration.

10. The method of claim 9, further comprising:
receiving, via a network interface, an indication identifying one or more misclassified treatment profiles and a corresponding ground-truth label for each misclassified treatment profile;
writing the indication and the ground-truth label for each misclassified treatment profile to an audit log data structure stored in the non-transitory memory; and
executing the refinement protocol using the audit log data structure to generate the updated diagnostic configuration.

11. The method of claim 9, wherein the initial diagnostic configuration comprises:
a first ruleset comprising a first set of marker values defining a first transition point for classifying a treatment profile into a first disease state; and
a second ruleset comprising a second set of marker values defining a second transition point for classifying a treatment profile into a second disease state,
wherein the first ruleset and the second ruleset are stored as versioned configuration records that are selectable by the diagnostic system at runtime.

12. The method of claim 11, wherein the refinement protocol comprises:
for each attribute, computing a marker deviation between the first set of marker values and the second set of marker values and storing the marker deviation in a deviation table comprising (i) an attribute identifier, (ii) a biomarker identifier, and (iii) a deviation value; and
generating the updated set of marker values by applying the marker deviations from the deviation table to produce an updated ruleset that, when executed by the diagnostic system, reduces a misclassification rate for the plurality of treatment profiles relative to the initial diagnostic configuration.

13. The method of claim 9, wherein the disease definition data comprises a first set of attributes associated with a first organization and a second set of attributes associated with a second organization; and
wherein determining the initial diagnostic configuration further comprises:
generating the initial diagnostic configuration by producing a merged configuration record that (i) maps each attribute to a selected marker value chosen from the first set of marker values or the second set of marker values and (ii) stores, for each selected marker value, provenance metadata identifying the organization from which the selected marker value originated.

14. The method of claim 13, wherein the refinement protocol comprises:
generating, for each treatment profile, a normalized biomarker feature vector comprising normalized expression levels corresponding to the attributes in the merged configuration record;
comparing each marker deviation to the normalized biomarker feature vectors across the plurality of treatment profiles to compute a precision metric for the merged configuration record; and
generating the updated set of marker values by modifying the merged configuration record until the precision metric satisfies a target precision threshold, and storing the updated diagnostic configuration as a new version of the merged configuration record.

15. The method of claim 9, wherein the refinement protocol comprises:
for each attribute, generating a time-series record of biomarker expression levels for at least a subset of the treatment profiles;
determining a change pattern by computing, from the time-series record, at least one rate-of-change value for the biomarker expression level over a defined time window; and
generating the updated set of marker values based on the rate-of-change value such that the updated diagnostic configuration classifies subsequent treatment profiles using both (i) a transition-point marker value and (ii) a change-pattern threshold derived from the rate-of-change value.

16. The method of claim 9, further comprising:
determining correlations between a subset of the attributes across the plurality of treatment profiles by generating a correlation matrix data structure storing correlation coefficients indexed by attribute identifiers;
identifying at least one different attribute based on correlation coefficients in the correlation matrix satisfying a selection criterion stored in memory;
updating the plurality of attributes by updating an attribute registry that defines which biomarkers are ingested into the normalized biomarker representation; and
in response to updating the attribute registry, regenerating the initial diagnostic configuration by generating a new versioned configuration record that includes marker values for the at least one different attribute.

17. A non-transitory computer-readable medium storing instructions thereon that, when executed by at least one processor, cause the at least one processor to:
receive disease definition data identifying a plurality of attributes associated with a disease, each attribute mapped to at least one biomarker;
generate, for each treatment profile of a plurality of treatment profiles, a normalized biomarker representation comprising quantified expression levels of the biomarkers;
determine an initial diagnostic configuration comprising a set of market values defining classification thresholds for the plurality of attributes;
execute a refinement protocol that iteratively evaluates misclassification of the treatment profiles by the initial diagnostic configuration by comparing the normalized biomarker representation to the classification thresholds;
in response to the refinement protocol, generate an updated diagnostic configuration comprising an updated set of market values that reduce misclassification relative to the initial diagnostic configuration; and
store the updated diagnostic configuration in a non-transitory memory accessible by the diagnostic system, wherein the diagnostic system is configured to classify subsequently received treatment profiles using the updated diagnostic configuration.

18. The non-transitory computer-readable medium of claim 17, further comprising instructions that cause the at least one processor to:
receive, via a network interface, an indication identifying one or more classified treatment profiles and a corresponding ground-truth label for each misclassified treatment profile;
write the indication, the ground-truth label for each misclassified treatment profile; and
execute the refinement protocol using the audit log data structure to generate the updated diagnostic configuration.

19. The non-transitory computer-readable medium of claim 17, wherein the initial diagnostic configuration comprises:
a first ruleset comprising a first set of marker values defining a first transition point for classifying a treatment profile into a first disease state; and
a second ruleset comprising a second set of marker values defining a second transition point for classifying a treatment profile into a second disease state,
wherein the first ruleset and the second ruleset are stored as versioned configuration records that are selectable by the diagnostic system at runtime.

20. The non-transitory computer-readable medium of claim 19, wherein the refinement protocol comprises:
for each attribute, computing a marker deviation between the first set of marker values and the second set of marker values and storing the marker deviation in a deviation table comprising (i) an attribute identifier, (ii) a biomarker identifier, and (iii) a deviation value; and
generating the updated set of marker values by applying the marker deviations from the deviation table to produce an updated ruleset that, when executed by the diagnostic system, reduces a misclassification rate for the plurality of treatment profiles relative to the initial diagnostic configuration.
